(19) United States
(12) Patent Application Publication
(10) Pub. No.: US 2002/0155083 A1
(43) Pub. Date: Oct. 24, 2002

(54) Dermal regeneration methods and compositions related thereto in conjunction with laser phototherapy, microdermabrasion and epidermabrasion

(76) Inventor: Morris Mann, Glendale, AZ (US)

Correspondence Address: The Halvorson Law Firm, 405 W. Southern Ave. Ste 1, Tempe, AZ, 85282, US

(21) Appl. No.: 10/109,253

(22) Filed: Mar. 26, 2002

Related U.S. Application Data
(60) Provisional application No. 60/285,100, filed on Apr. 19, 2001.

(51) Int. Cl. A61K 38/17; A61K 7/06; A61K 7/11

(52) U.S. Cl. 424/70.14; 514/12; 132/209

(57) ABSTRACT

The present invention generally relates to compositions and methods for dermal regeneration, both alone and in conjunction with LASER phototherapy. The method comprises the steps of applying an abrasive scrub to a selected area of a dermis to be treated to remove keratin from an epidermis; applying a keratin solvent system to the selected area of the dermis to be treated after the step of applying an abrasive scrub to further remove keratin from the epidermis; applying an acid chemical peel composition to the area of the dermis to be treated; and applying a thymic peptide based composition on the area of the dermis. A LASER phototherapy treatment may be substituted or used in conjunction with the acid chemical peel treatment. Additionally, microdermabrasion or epidermabrasion may be used prior to the chemical peel.

### FIELD OF THE INVENTION

The present invention generally relates to compositions and methods for dermal regeneration, both alone and in conjunction with LASER phototherapy, microdermabrasion and/or epidermabrasion.

### BACKGROUND OF THE INVENTION

Skin aging is a natural phenomenon associated with degenerative structural changes in the epidermis, such as decrease in the level of collagen in the dermis. It has also been found that skin aging can be accelerated by factors in the environment, such as exposure to UV radiation and smoking. Skin aging, and the attendant appearance of advancing age, has always been considered undesirable by certain portions of the population. Therefore, there is a clear need in the art for methods and compositions that will induce dermal regeneration.

Many techniques to decrease the appearance of aging have been developed, such as the use of cosmetics, surgery, LASERs, and/or chemical means. Lately, chemical peels and LASER phototherapy have been used to promote dermal regeneration. Further, it has been found that the application of LASER phototherapy in conjunction with the method as described herein produces more dermal collagen proliferation than either phototherapy or chemical methods alone. In addition, it has been noted that that the chemical treatment described herein reduces the power required to induce changes using LASER phototherapy alone.

### SUMMARY OF THE INVENTION

An object of the present invention is to induce maximal dermal regeneration and, to the extent possible, maintain the integrity of the epidermis. This is because medical and cosmetic post-procedure recovery time is far shorter if epidermal integrity is maintained. Therefore, an individual can have the procedure performed and appear in public the following day without attracting a great deal of unwanted attention and embarrassment. Consequently, the methodology according to the present invention can be applied more frequently than other currently utilized approaches. Since the effects are additive, the effects achieved in terms of skin regeneration and attendant youthfulness are more substantial. Additionally, maintaining the integrity of the epidermis minimizes the risk of post-procedure inflammation and/or infection.

There are four compositions, applied in steps, which constitute the method of the present invention. They are: 1) an abrasive scrub, 2) a keratolytic solvent, 3) a physiologic chemical peel, and 4) a thymic peptide based cream to be applied immediately following the procedure and in subsequent days as an aftercare adjunct. These compositions and their method of use will be described more completely below.

The use of LASER phototherapy immediately after the application of the physiologic chemical peel results in more proliferation of collagen in the dermis than either the method of the present invention or LASER phototherapy alone. In addition, subsequent application of the thymic fraction 5 peptide emollient results in substantially less epidermal inflammation and tissue destruction. Further, application of the fraction 5 peptide emollient also results in enhanced fibroblastic activity, thereby speeding recovery.

Unlike laser phototherapy, it was found that both microdermabrasion and epidermabrasion were most effective in conjunction with the described chemical rejuvenation system when done after the application of the area gel immediately prior to the use of the chemical peel. However, as with laser phototherapy, the fraction 5 emollient induces enhanced fibroblastic activity, more rapid recovery, and less attendant epidermal inflammation.

The novel features that are considered characteristic of the invention are set forth with particularity in the appended claims. The invention itself, however, both as to its structure and its operation together with the additional object and advantages thereof will best be understood from the following description of the preferred embodiment of the present invention when read in conjunction with the accompanying drawings. Unless specifically noted, it is intended that the words and phrases in the specification and claims be given the ordinary and accustomed meaning to those of ordinary skill in the applicable art or arts. If any other meaning is intended, the specification will specifically state that a special meaning is being applied to a word or phrase. Likewise, the use of the words “function” or “means” in the Description of Preferred Embodiments is not intended to indicate a desire to invoke the special provision of 35 U.S.C. §112, paragraph 6 to define the invention. To the contrary, if the provisions of 35 U.S.C. §112, paragraph 6, are sought to be invoked to define the invention(s), the claims will specifically state the phrases “means for” or “step for” and a function, without also reciting in such phrases any structure, material, or act in support of the function. Even when the claims recite a “means for” or “step for” performing a function, if they also recite any structure, material or acts in support of that means of step, then the intention is not to invoke the provisions of 35 U.S.C. §112, paragraph 6. Moreover, even if the provisions of 35 U.S.C. §112, paragraph 6, are invoked to define the inventions, it is intended that the inventions not be limited only to the specific structure, material or acts that are described in the preferred embodiments, but in addition, include any and all structures, materials or acts that perform the claimed function, along with any and all known or later-developed equivalent structures, materials or acts for performing the claimed function.

### DESCRIPTION OF THE PREFERRED EMBODIMENTS

The method and composition according to the present invention are useful for the induction of dermal regeneration.

A first part of the method consists of the application of an abrasive scrub to the area of the dermis to be treated. Obviously, many abrasive scrubs have been formulated, as evidenced in the prior art. Therefore, it should be noted that the novelty is not in the formulation of the scrub per se but in the use of an abrasive scrub as the first step in the regenerative method as a whole. In the first step of the method according to the present invention, the abrasive scrub is applied to the area of the skin surface that is targeted for rejuvenation. One preferred abrasive scrub formula is disclosed in the method is described in Table 1, below. Obviously, one skilled in the art could choose other abrasive scrub formulations without altering the spirit of the invention. However, it is necessary that the scrub used remove keratin from the epidermis, thereby facilitating the remaining steps in the method.

The second step in the method is the application for a period of time of a keratin solvent system to the area of the dermis to be treated. The purpose of this step is, once again, to remove the keratin in the epidermis, thereby facilitating penetration into the dermis through the epidermis of substances that will produce dermal regeneration.

Keratin solvent systems include either urea, which can effectively solubilize keratin, or acetic acid based solutions that will also solubilize keratin. However, it should be noted that in practice there is a margin of safety with urea that is not present with acetic acid based solutions. Therefore, a urea-based solution has been used more extensively in practice. Preferred, keratin solvent systems of the present invention comprise urea gel formulations, hyporeacitve urea gel formulations, and also acetic acid based solutions. The keratin solution representative formulas and ranges are noted in Tables 2A and 2B, below.

The chosen keratin solvent system is applied to the area of the skin where dermal regeneration is desired for a selected period of time ranging from 5 minutes to 6 hours or more.

Optionally, at this time a second application of the abrasive scrub, according to the first step of the method may be applied. This optional additional step, which would be chosen by the physician carrying out this method, removes even more keratin to further enhance penetration of an acid peel composition through the epidermis.

In this optional step, the abrasive scrub is preferably microdermabrasion or epidemabrasion. Microdermabrasion, which implies the use of very fine particles blown onto the skin surface is a new technique that is being used to reduce or eliminate fine wrinkles on the skins surface. Results utilizing the technology for chemical regeneration described herein in conjunction with microdermabrasion is better than either technology alone for rejuvenating the skin.

Guided epidermabrasion implies the use of paper, plastic or any other background substance coated with a fine abrasive material that can be manipulated by an individual to smooth the skin in the same way that sandpaper is used to smooth wood. Obviously, this technique can be done manually or with an appropriate mechanical device. Once again, the combination of this technique with the technology for chemical regeneration described there is better than either technology alone for rejuvenating the skin.

Microdermabrasion, as well as epdermabrasion, when used prior to the administration of the acid peel result in significantly greater penetration of the acid peel. This in turn results in more substantial rejuvenation as noted by histological analysis wherein fibroblastic activity is significantly increased as is dermal collagen production.

The third step of the method according to the present invention is the application of an acid chemical peel solution to the skin area targeted for dermal regeneration. The acid peel composition consists of a series of physiologic acids, including, but not limited to, citric acid and pyruvic acid, which are krebs's cycle intermediaries, in conjunction with a surfactant that will enhance penetration of the chemical peel solution into the dermis and a solubilizing system, which also facilitates penetration. The acid chemical peel composition may remain on the skin surface for selected time periods, ranging between 1 minute to 8 hours or more. Further penetration of the dermis by the acid chemical peel composition can be achieved by occluding the treated skin, after application of the acid chemical peel composition, with a thin polymeric film such as plastic kitchen wrap or some other occlusive material. A representative acid peel system with ranges is noted in Table 3, below.

The fourth step in the method according to the present invention is after application of the acid chemical peel composition, the area of the dermis being treated is subjected to treatment with a LASER treatment system. Preferably, the LASER treatment system is one that uses LASER light at wavelengths between 450 nm and 1400 nm. It should be recognized that steps three and four, the acid chemical peel and LASER phototherapy described above, may be performed individually or in combination and still fall within the scope of the present invention.

Histological specimens retrieved from subjects following only the application of an acid chemical peel solution have shown definitive increases in collagen and fibroblastic proliferation. However, use of an acid chemical peel in conjunction with LASER treatment produces far greater dermal regeneration than either technology used alone. Specifically, it has been noted that following treatment with the acid chemical peel, immediate subsequent treatment with LASER light at wavelengths of between 450 nm and 1400 nm produce far more collagen generation and fibroblastic proliferation than would be the case with either technology alone. This is important since the increased collagen generation and fibroblastic proliferation produce a more desirable final cosmetic outcome.

The fifth step in the method of the present invention is the application of a thymic peptide based cream immediately following the acid chemical peel and LASER procedures. The patient applies this same thymic peptide based cream on subsequent days as an aftercare adjunct to speed the healing process, decrease inflammation, and reduce the risk of infection.

One skilled in the art will recognize that one of the untoward effects associated with either chemical peels or LASER phototherapy, and especially both in combination, is intense epidermal inflammation and even epidermolysis. Because one role of the epidermis is to provide a barrier against infection, any disruption in the integrity of the epidermis necessarily makes the patient more susceptible to infection by a multitude of microorganisms. This problem is greatly ameliorated by the use of a Thymosin Fraction 5 (TF5) based emollient after the acid chemical peel and/or after LASER phototherapy. Surprisingly, even after applying the acid chemical peel and the LASER phototherapy the TF5 based emollient will greatly diminish the inflammation and epidermolysis that would otherwise occur. However, the amount of TF5 based emollient must be increased substantially over the amount currently used by those skilled in the art in order to produce the desired effect.

TF5 is a partially purified mixture of polypeptides that is routinely prepared from calf thymus glands. However, TF5 may also be prepared from porcine, ovine, murine, goat, rat chicken and human thymus tissues. Preparation and isolation of TF5 has been described (Hooper et al., “The purification and properties of bovine thymosin,” Ann. NY Acad. Sci. 249:125, 1975). TF5 consists of at least 40 to 50 distinct polypeptides separated by isoelectric focusing on polyacrylamide gel plates (pH 3.5-9.5). TF5 is essentially free of lipids, carbohydrates and endotoxins. TF5 has previously been demonstrated to be effective in reconstituting immune functions in thymic-deprived or immunodeprived animals, in humans with primary immunodeficiencies, and in immunosuppressed cancer patients. An apparent primary effect of this mixture of peptides is to stimulate cell-medicated immunity.

Two of the major biologically active ingredients in TF5 are thymosin α₁ (Tα₁) an immunomodulatory peptide of 28 amino acids (molecular weight 3,108 daltons) (Low et al., “The chemistry and biology of Thymosin I. isolation and characterization and biological activities of Tα₁ and polypeptide β₁ from calf thymus,” J. Bio. Chem. 254:981, 1979) and thymosin β₄ (Tβ₄), an actin-sequestering peptide of 43 amino acids (molecular weight 4,963 daltons) (Low, T. L. K., and Goldstein, A. L., “Chemical characterization of thymosin β₄,” J. Bio. Chem. 257:1000, 1982). Tα₁ and Tβ₄ are highly conserved in nature and their amino acid sequences are identical in most mammalian species.

More than a dozen TF5-like preparations have been prepared from calf or porcine thymus tissue. These thymic extracts such as thymostimulin (TP-1), TFX, thymalin, thymoject, thym-Uvocal, and others, are variations of the TF5 formulation and are all partially purified preparations composed primarily of polypeptide mixtures with molecular weights of 15,000 or less. The major biologically active components of TF5 contain Tα₁ and Tβ₄, as well as lower concentrations of other purified will characterized thymosin peptides such as prothymosin a (Pro Tα₁), Tα₂ to Tα₁₁ and Tβ₃, Tβ to Tβ₁₃, MB 35, MB 40, ubiquitin, thymulin (FTS), thymic humoral factor (THFα₂) and thymopoietin (TP). The TF5-like extracts prepared by variations of the procedure used originally to prepare TF5 may also contain α and β as key ingredients and smaller quantities of the other peptides described in TF5 such as Pro Tα₃, FTS, THFαC₂, TP, ubiquitin and MB 35 and MB 40.

The method of the present invention preferably includes a formulation of TF5 and TF5-like thymic peptide extracts that contain Tα₁ and/or Tβ₄. Tα₁ and Tβ₄ have been characterized with regard to their ability to stimulate and regulate cell-mediated immunity, to enhance wound healing, and for their ability to increase resistance to microbial infections and to decrease microbial adherence (Baumann et al., Preclinical studies of thymosin α₁ and thymosin β₄, In: Mauer, H. R., Goldstein, A. L., Hager, E. D., Thymic peptides in preclinical and clinical medicine, W. Zuckschwerdt Verlag Munchen, Bern, Wien, New Your, pp.13-17, 1977). The terms TF5 or TF5-like, as used in the present application, refer only to those thymic extracts that include Tα₁ and/or Tβ₄. Although other peptides may be present in some concentration in TF5 or TF5-like preparations, the presence of Tα₁ and/or T₄ in the TF5 or TF5-like preparation is required for use in the present invention, and preferably at a concentration ranging from 0.05 to 0.1% by weight for each Tα₁ and/or Tα₄.

The importance of TF5 or TF5-like preparations in preventing infections in <¢ immunocompromised patients was first suggested by studies in immunosuppressed animals (Oates, K., Goldstein, A. L., Thymosin. In De Vita, D. T. Hellman, S., Rosenberg, S. A. (eds.), Biological therapy of cancer, 2^{nd} ed., J B Lippencott, Philadelphia, pp. 705-718, 1995; Goldstein, A. L., “Clinical applications of thymosin alpha-1,” Cancer Invest. 12:545-547, 1994). Early studies demonstrated an increased survival rate of immunosuppressed mice infected with BCO, Candida, or Cryptococcus when these animals were treated with TF5, Tα₁ or TF5-like preparations (Collins, F. M. and Morrison, N. E., “Restoration of T-cell responsiveness by thymosin: Expression of antituberculosis immunity in mouse lungs,” Infect. Immun. 23:330, 1979; Bistoni et al., “Increase of mouse resistance to Candida albicans infection by Thymosis α₁,” Infect. Immun. 36(2): 609-614, 1982). In T-cell depleted mice, TF5 has been found to restore cellular immunity to Blastomyces dermatitidis (Longley, R. E. and Cozad, G. C., “Thymosin restoration of cellular immunity to Blastomyces dermatitidis in T-cell depleted mice,” Infect. Immun. 26(1):187-92, 1979). In similar studies cell-mediated immunity to Listeria monocytogenes was increased in protein malnourished mice following treatment with TF5. The administration of TF5 or Tα₁ also stimulated a significant rise in the amount of interferon (IFN) produced in mice infected with the Newcastle disease virus (Huang et al., “Thymosin treatment modulates production of interferon,” J. Interferon Res. 1:411, 1981). In similar studies in mice, an injection of TF5 or Tα₁ increased resistance to infection with Candida albicans (Bistoni et al., “increase of mouse resistance to Candida albicans infection by Thymosin α₁,” Infect. Immun. 36(2):609-614, 1982; Salvin, S. B. and Neta, R., “Resistance and susceptibility to infection in inbred murine strains. I. Variations in the response to thymic hormones in mice infected with Candida albicans,” Cell Immunol. 75:160, 1983). The increased resistance to infection with an infections agent after administration of TF5 has been attributed to an increase in the release of specific cytokines such as MIF and IFN (Salvin, S. B. and Neta, R., “Resistance and susceptibility to infection in inbred murine strains. I. Variations in the response to thymic hormones in mice infected with Candida albicans,” Cell Immunol. 75:160, 1983). Injection of TF5 and Tα1 has also been shown to protect 5-fluorouracil (5FU)- or morphine-immunosuppressed mice against opportunistic infections with C. albicans, Listeria monocytogenes, Pseudomonas aeruginosa, and Serratia marescens (Ishitsuka et al., “Protective activity of thymosin against opportunistic infections in animal models,” Cancer Immunol. Immunother. 14:145, 1983; Di Francesco et al., “Combined effect of flucazole and thymosin α₁ on systemic candidiasis in mice immunosuppressed by morphine treatments,” Clin. Ex. Immuno. 97:347-352, 1994). The efficacy of Tα₁ administered in combination with the antiviral drug amantadine and interferon was also demonstrated in mice infected with the influenza virus. This new combination therapy protocol has been found to significantly increase the long term survival, to reduce viral titers in the lungs, and to restore a number of the immunological parameters tested such as natural killer cell activity, cytotoxic T-lymphocyte responses, and subsets of CD4+/CD8+ lymphocytes (D'Agostini et al., “Efficacy of combination therapy with amantadine, Tα₁ and α/β IFN in mice infected with influenza A virus,” Int. J. Immunopharmacol. 18:95-102, 1996).

Clinical studies in humans using various TF5 or TF5-like preparations (e.g., THF, TFX, TP-I) have shown that the administration of thymic fractions can shorten the course of viral infections (e.g., herpes zoster, herpes simplex, adenovirus, hepatitis, and icytomegalovirus) and increase the restoration of T-cell immunity in these patients (Ajiuti et al., “A placebo controlled trial of thymic hormone treatment of recurrent herpes simplex labialis infection in immunodeficient host: Results after a 1 year follow up,” Clin. Immunol. Immunopathol. 30:11, 1984; Businco, L. and Rezza, E., “Therapy of viral disease in immunosuppressed patients with TP-1,” Thymic Hormones and T-lymphocytes (A. F. Wigzel, ed.), Academic Press, New York, p. 295, 1981; Demartino et al., “Tlymphocytes in children with respiratory infections: Effect of the use of thymostimulin on the alteration of T-cell subsets,” Int. J. Tissue React. 6:223, 1984; Schulof, R. S. and Goldstein, A. L., “Clinical applications of thymosin and other thymic hormones,” Recent Advances in Clinical Immunology (R. A. Thompson and N. R. Rose, eds.), Churchill Livingstone, Edinburgh, p. 243, 1983; Trianin et al., “The role of THF a thymic hormone, as a regulator of T-cell differentiation in humans,” Current Concepts in Human Immunology and Cancer Immunomodulation (Serrpu et al., eds.), Elsevier Biomedical, New York, p. 295, 1981). These studies suggest that TF5 or TF5-like preparations may be useful in preventing or attenuating infections in immunocompromised hosts.

TF5 and TF5-like preparations have also been utilized, in clinical trials, in patients with primary and secondary immunodeficiencies. A number of syndromes related to congenital defects of the immune system are included in this category. These may include T-cell, B-cell or both lymphocyte population defects. An increase in the percentage and numbers of Erosette-forming cells in vitro after incubation with TF5 and TF5-like preparations has been reported (Schulof, R. S. and Goldstein, A. L., “Clinical applications of thymosisn and other thymic hormones,” Recent Advances in Clinical Immunology (R. A. Thompson and N. R. Rose, eds.), Churchill Livingstone, Edinburgh, p. 243, 1983).

Several thymic preparations, such as TF5, TP-1, TP-5, thymulin, THF, and TPX, have been studied in clinical trials in children with primary immunodeficiency diseases (Goldstein, A. L., “Clinical applications of thymosin α₁,” Cancer Invest. 12:545-547, 1994, Trianin et al., “The role of THF a thymic hormone, as a regulator of T-cell differentiation in humans,” Current concepts in Human Immunology and Cancer Immunomodulation (Serrpu et al., eds.), Elsevier Biomedical, New York, p. 295, 1981; Bach, J. F. and Dardenne, M. “Clinical aspects of thymulin (FTS),” Thymic Hormones and Lymphokines (A. L. Goldstein, ed.), Plenum Press, New York, p. 593, 1984; Davies, E. G. and Levinsky, R. J., “Experience in the use of thymic hormones for immunodeficiency disorders,” Thymic Factor Therapy, Vol. 16 (N. A. Byron and J. R. Hobbs, eds.), Serono Symposium Publications, Raven Press, New York, p. 156, 1984; Goldstein, A. L., “thymosin alph-1: [Note: “alph-1” is in original copy.] Chemistry, mechanism of action and clinical applications,” Combination Therapies 2 (E. Garaci and A. L. Goldstein, eds.), Plenum Press, New York, pp. 39-48, 1993; Skotnicki et al., “Biological properties and clinical use of calf thymus extract TFX-Polfa,” Thymic Hormones and Lymphokines (A. L. Goldstein, ed.), Plenum Press, New York, p. 545, 1984; Wara et al., “Thymosin fraction 5 therapy in patients with primary immunodeficiency disorders,” Thymic Factor Therapy, Vol. 16 (N. A. Buron and J. R. Hobbs, eds.), Serono Symposia Publications, Raven Press New York, p. 123, 1984). These studies indicate that thymic fractions are useful in reconstituting cellular immune responses and improving clinical status in these patients.

The Tα₁ fraction and the Tβ fraction have been completely characterized and are noted for their ability to decrease microbial adherence, enhance wound healing, and stimulate cell-mediated immunity. Thus, topical application of these particular thymic fractions increase the ability of the skin to resist fungal and other infections, and appear to assist the skin's ability to mount a reaction to fungus and eliminate it. Therefore, TF5 not only enhances healing but also prevents the possibility of infection.

A typical formula for the TF5 based emollient with ranges is noted in Table 4, below. One skilled in the art will note that the amount of TF5 has been increased in this formulation to provide the benefits described above.

In addition to the other components noted in the formal for the thymosin fraction 5 emollient, it was determined that an anti-inflammatory substance with a concentration range of 0.001 to 85% (preferably 1.0%) would be a useful adjunct. Many anti-inflammatory substances are potentially effective. These include, but are not limited to, non-steroidial anti-inflammatory drugs such as ibuprofen, aspirin, -α₁ bisubolol, trisoleoyltromethanamine ethane sulphate (PX-13), and the like.

The method herein described consisting of the four noted compositions with or without LASER phototherapy dramatically enhances dermal proliferation of collagen with minimal epidermal damage.

The preferred embodiment of the invention is described above in the Drawings and Description of Preferred Embodiments. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s). The foregoing description of a preferred embodiment and best mode of the invention known to the applicant at the time of filing the application has been presented and is intended for the purposes of illustration and description. It is not intended to be exhaustive or to limit the invention to the precise form disclosed, and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated.

What is claimed is:

1) A method for the regeneration of dermal tissue comprising the steps of:
a) applying an abrasive scrub to a selected area of a dermis to be treated to remove keratin from an epidermis;
b) applying a keratin solvent system to the selected area of the dermis to be treated after the step of applying an abrasive scrub to further remove keratin from the epidermis;
c) applying an acid chemical peel composition to the area of the dermis to be treated; and
d) applying a thymic peptide based composition on the area of the dermis.

2) The method according to claim 1 further including the step of applying LASER phototherapy to the area of the dermis to be treated after the step of applying the acid chemical peel to the area to be treated, but before the step of applying the thymic based composition.

3) The method according to claim 2 further including an additional step of applying a second application of the abrasive scrub, comprising epidermabrasion or microdermabrasion, to the area to be treated before the step of applying the acid chemical peel composition.

4) The method according to claim 3 wherein the thymic based composition comprises a Thymosin Fraction 5 composition and an anti-inflammatory composition.

5) The method according to claim 3 wherein the thymic based composition comprises a Thymosin Fraction 5 composition and an anti-inflammatory composition.

6) The method according to claim 1 wherein the keratin solvent systems includes either urea, which can effectively solubilize keratin, or an acetic acid based solution that will also solubilize keratin.

7) The method according to claim 1 wherein the acid chemical peel composition comprises a series of physiologic acids, which are krebs's cycle intermediaries, in conjunction with a surfactant, which enhances penetration of the chemical peel solution into the dermis, and a solubilizing system, which also facilitates penetration of the chemical peel solution into the dermis.

8) The method according to claim 6 wherein the thymic based composition comprises a Thymosin Fraction 5 composition and an anti-inflammatory composition.

9) The method according to claim 7 wherein the thymic based composition comprises a Thymosin Fraction 5 composition and an anti-inflammatory composition.

10) A method for the regeneration of dermal tissue comprising the steps of:
a) applying an abrasive scrub to a selected area of a dermis to be treated to remove keratin from an epidermis;
b) applying a keratin solvent system to the selected area of the dermis to be treated after the step of applying an abrasive scrub to further remove keratin from the epidermis;
c) applying a LASER phototherapy treatment to the area of the dermis to be treated; and
d) applying a thymic peptide based composition on the area of the dermis.

11) The method according to claim 10 further including the step of applying an acid chemical peel composition to the area of the dermis to be treated before the step of applying the LASER phototherapy to the area to be treated, but before the step of applying the thymic based composition.

12) The method according to claim 11 further including an additional step of applying a second application of the abrasive scrub, comprising epidermabrasion or microdermabrasion, to the area to be treated before the step of applying the LASER phototherapy treatment.

13) The method according to claim 12 wherein the thymic based composition comprises a Thymosin Fraction 5 composition and an anti-inflammatory composition.

14) The method according to claim 13 wherein the thymic based composition comprises a Thymosin Fraction 5 composition and an anti-inflammatory composition.

15) The method according to claim 10 wherein the k eratin solvent systems includes either urea, which can effectively solubilize keratin, or an acetic acid based solution that will also solubilize keratin.

16) The method according to claim 11 wherein the acid chemical peel composition comprises a series of physiologic acids, which are krebs's cycle intermediaries, in conjunction with a surfactant, which enhances penetration of the chemical peel solution into the dermis, and a solubilizing system, which also facilitates penetration of the chemical peel solution into the dermis.

17) The method according to claim 15 wherein the thymic based composition comprises a Thymosin Fraction 5 composition and an anti-inflammatory composition.

18) The method according to claim 16 wherein the thymic based composition comprises a Thymosin Fraction 5 composition and an anti-inflammatory composition.

19) A topical composition for the regeneration of dermal tissue comprising:
a) Thymosin Fraction 5, ranging from 0.001-55 percent by weight;
b) Anti-inflammatory compound, ranging from 0.001-85 percent by weight;
c) Deionized water, ranging from 0.0-95 percent by weight;
d) Arlamol, ranging from 0.0-80 percent by weight;
e) Glycerin, ranging from 0.0-95 percent by weight;
f) Squalane, ranging from 0.0-90 percent by weight;
g) Jewelweed, ranging from 0.0-90 percent by weight;
h) Germaben II, ranging from 0.0-25 percent by weight;
i) Caropol Ultrez 10, ranging from 0.0-50 percent by weight;
j) Panthenol, ranging from 0.0-50 percent by weight;
k) Allantoin, ranging from 0.0-50 percent by weight;
l) Guar, ranging from 0.0-80 percent by weight;
m) Methyl Paraben, ranging from 0.0-25 percent by weight;
n) Kathon, ranging from 0.0-25 percent by weight;
o) Alpha-Bisabolol, ranging from 0.0-50 percent by weight; and
p) Curdlan, ranging from 0.0-25 percent by weight.

20) The topical composition of claim 19 wherein the
a) Thymosin Fraction 5, is approximately 1.0 percent by weight;
b) Anti-inflammatory compound, is approximately 1.0 percent by weight;
c) Deionized water, is approximately 85.7 percent by weight;
d) Arlamol, is approximately 5.0 percent by weight;
e) Glycerin, is approximately 1.5 percent by weight;
f) Squalane, is approximately 1.0 percent by weight;
g) Jewelweed, is approximately 1.0 percent by weight;
h) Germaben II, is approximately 1.0 percent by weight;
i) Caropol Ultrez 10, is approximately 0.8 percent by weight;
j) Panthenol, is approximately 0.5 percent by weight;
k) Allantoin, is approximately 0.5 percent by weight;
l) Guar, is approximately 0.5 percent by weight;
m) Methyl Paraben, is approximately 0.3 percent by weight;
n) Kathon, is approximately 0.1 percent by weight;
o) Alpha-Bisabolol, is approximately 0.1 percent by weight; and
p) Curdlan, is approximately 0.01 percent by weight.
